(19) United States
(12) Patent Application Publication
(10) Pub. No.: US 2004/0157252 A1
(43) Pub. Date: Aug. 12, 2004

(54) Methods for transcription detection and analysis

(75) Inventor: Carsten Rosenow, Redwood City, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/763,614

(22) Filed: Jan. 23, 2004

Related U.S. Application Data
(63) Continuation of application No. 09/683,710, filed on Feb. 5, 2002.
(60) Provisional application No. 60/266,718, filed on Feb. 5, 2001.

(51) Int. Cl. C12Q 1/68

(52) U.S. Cl. 435/6

(57) ABSTRACT

In one aspect of the invention, methods for discovering transcripts are provided. In one embodiment, oligonucleotide probe arrays are used to interrogate the genome for transcription. Methods are also provided for identifying potential operon elements, 5″ and 3″ UTR sequences.

### CROSS REFERENCE TO RELATED APPLICATIONS

The present inventors claim priority to U.S. Provisional No. 60/266,718, filed Feb. 2, 2001, which is hereby incorporated by reference in its entirety for all purposes.

### BACKGROUND OF INVENTION

The present invention relates genetic analysis and bioinformatics. Specifically, it discloses use of DNA microarrays to identify new transcripts in E. coli.

Genome sequence information has accumulated at a fast pace in recent years and the generation of whole genome sequences is now commonplace. However, the number of uncompleted genome projects significantly exceeds the number of completely annotated and published sequences. One of the primary reasons for this gap between sequence generation and the public release is the still difficult task of sequence annotation, of interpreting raw sequence data into useful biological information. Most of the genome annotation information is generated using bioinformatic approaches. These in silico methods used for gene predictions in combination with homology searches are applied to the primary genome sequence. However, the prediction of untranslated transcripts along with transcriptional start sites, promoter and terminator locations, and the precise boundaries of protein-coding regions within a genome are still subject to substantial uncertainty and often lack experimental support.

### SUMMARY OF INVENTION

In one aspect of the invention, methods are provided for detecting a transcribed genomic region. The methods include providing a nucleic acid sample containing transcripts or nucleic acids dervied from transcripts from the genome; hybridizing the nucleic acid sample with a plurality of nucleic acid probes, where the probes are designed to interogate potential transcripts from both strands of the genomic DNA; and analyzing hybridization signals to detect the transcribed region.

In some embodiments, the pluarlity of probes comprises probes interogating the intergenic, and intronic regions of the genome. The probes may be immobilized, on a substrate at a density greater than 400 or 1000 different probes per cm².

In another aspect of the invention, methods are provided for detecting an operon element in a prokaryote. The methods include hybridizing transcripts or nucleic acids dervied from transcripts from the organism with a plurality of probes, where the probes interrogate transcription of an intergenic region between two flanking open reading frames (ORFs); and classifying the intergenic region as a potential operon element if both flanking ORFs are expressed and if the intergenic region is transcribed off the same DNA strand as the flanking ORFs.

In some embodiments, the methods include classifying the intergenic region as operon element if both flanking ORFs are expressed and if the intergenic region is transcribed off the same DNA strand as the flanking ORFs and if transcription in the intergenic region is detected by more than 60% or 80% of the probes targeting the intergenic region.

In some preferred embodiments, method include classifying the intergenic region as a potential operon element if both flanking ORFs are expressed and if the intergenic region is transcribed off the same DNA strand as the flanking ORFs and the transcription of the intergenic region is correlated with the transcription of at least one of the flanking ORFs.

In yet another aspect of the invention, methods for detecting untranslated region (UTR) for a gene are provided. The methods include hybridizing a sample containing transcripts or nucleic acids dervied from transcripts with a plurality of probes, where the probes interrogate transcription of an intergenic region immediately upstream the gene; and classifying the intergenic region as a potential 5″UTR of the gene if the intergenic region is transcribed in the same orientation of the gene and the trancribed region is greater than 70 bases in length. Similarly, an intergenic region is classified as a potential 3″UTR of the gene if the intergenic region is transcribed in the same orientation of the gene, it is immediately downstream of the gene and the trancribed region is greater than 70 bases In length.

### BRIEF DESCRIPTION OF DRAWINGS

The accompanying drawings, which are incorporated in and form a part of this specification, illustrate embodiments of the invention and, together with the description, serve to explain the embodiments of the invention:

FIG. 1 shows operon detection using oligonucleotide probe intensities. Individual oligonucleotide probe intensities (PM MM) from three conditions to validate the microarray predicted hnr-galU operon. Intensities for individual probes interrogating hnr, the 200 bp Ig region and sulA are shown. This operon was independently confirmed using RT-PCR (data not shown).

FIG. 2 shows 5″ UTR detection upstream of opmA. Individual oligonucleotide probe intensities (PM MM) from three conditions to validate the microarray detected 5″ UTR upstream of ompA (22). Intensities for individual oligonucleotide probes interrogating ompA, the 356 bp Ig region and galU are shown. The arrows above the indicated genes show the direction of transcription

### DETAILED DESCRIPTION

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention. For example, various aspects of the invention are described using exemplary embodiments for synthesizing oligonucleotide probe arrays. The scope of the invention, however, is not limited to the synthesis of oligonucleotide probe arrays. For example, methods of the invention are also useful for synthesizing or immobilizing other polymers such as peptides.

All cited references, including patent and non-patent literature, are incorporated herein by reference in their entireties for all purposes.

General

As used in the specification and claims, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques of organic chemistry, polymer technology, molecular biology (including recombinant techniques), cell biology, biochemistry, and immunology, which are within the skill of the art. Such conventional techniques include polymer array synthesis, hybridization, ligation, detection of hybridization using a label. Such conventional techniques can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), all of which are herein incorporated in their entirety by reference for all purposes.

Additional methods and techniques applicable to array synthesis have been described in U.S. Pat. Nos. 5,143,854, 5,242,974, 5,252,743, 5,324,633, 5,384,261, 5,405,783, 5,412,087, 5,424,186, 5,445,934, 5,451,683, 5,482,867, 5,489,678, 5,491,074, 5,510,270, 5,527,681, 5,550,215, 5,571,639, 5,578,832, 5,593,839, 5,599,695, 5,624,711, 5,631,734, 5,677,195, 5,744,101, 5,744,305, 5,770,456, 5,795,716, 5,800,992, 5,831,070, 5,837,832, 5,856,101, 5,871,928, 5,858,659, 5,936,324, 5,968,740, 5,974,164, 5,981,185, 5,981,956, 6,025,601, 6,033,860, 6,040,138, and 6,090,555, which are all incorporated herein by reference in their entirety for all purposes.

Analogue when used in conjunction with a biomonomer or a biopolymer refers to natural and un-natural variants of the particular biomonomer or biopolymer. For example, a nucleotide analogue includes inosine and dideoxynucleotides. A nucleic acid analogue includes peptide nucleic acids. The foregoing is not intended to be exhaustive but rather representative. More information can be found in U.S. Pat. No. 6,156,501.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% or 95%, and more preferably from about 98 to 100%. Alternatively, substantial complementarity exists when an RNA or DNA strand will hybridize under selective hybridization conditions to its complement. Typically, selective hybridization will occur when there is at least about 65% complementarity over a stretch of at least 14 to 25 nucleotides, preferably at least about 75%, more preferably at least about 90% complementarity. See e.g., M. Kanehisa Nucleic Acids Res. 12:203 (1984), incorporated herein by reference.

Hybridization refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25° C. For example, conditions of 5× SSPE (750NaCl, 50NaPhosphate, 5EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2 nd Ed. Cold Spring Harbor Press (1989) which is hereby incorporated by reference in its entirety for all purposes above.

Nucleic acid refers to a polymeric form of nucleotides of any length, such as oligonucleotides or polynucleotides, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be customized to stabilize or destabilize hybrid formation or enhance the specificity of hybridization with a complementary nucleic acid sequence as desired.

Oligonucleotide or polynucleotide is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Sample Preparation

In one aspect of the invention, methods are provided for analyzing transcripts from a genome and for drug discovery. In some preferred embodiments, a biological sample from cells of the species of interest (the species whose genome is to be annotated, for example, E. coli., yeast, dog or human) is obtained and a nucleic acid sample is prepared and analyzed.

One of skill in the art will appreciate that it is desirable to have nucleic samples containing target nucleic acid sequences that reflect the transcripts of the cells of interest. Therefore, suitable nucleic acid samples may contain transcripts of interest. Suitable nucleic acid samples, however, may contain nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like. Transcripts, as used herein, may include, but not limited to pre-mRNA nascent transcript(s), transcript processing intermediates, mature mRNA(s) and degradation products.

In one embodiment, such sample is a homogenate of cells or tissues or other biological samples. Preferably, such sample is a total RNA preparation of a biological sample. More preferably in some embodiments, such a nucleic acid sample is the total mRNA isolated from a biological sample. Those of skill in the art will appreciate that the total mRNA prepared with most methods includes not only the mature mRNA, but also the RNA processing intermediates and nascent pre-mRNA transcripts. For example, total mRNA purified with poly (T) column contains RNA molecules with poly (A) tails. Those poly A+ RNA molecules could be mature mRNA, RNA processing intermediates, nascent transcripts or degradation intermediates.

Biological samples may be of any biological tissue or fluid or cells. Typical samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

Another typical source of biological samples are cell cultures where gene expression states can be manipulated to explore the relationship among genes.

One of skill in the art would appreciate that it is desirable to inhibit or destroy RNase present in homogenates before homogenates can be used for hybridization. Methods of inhibiting or destroying nucleases are well known in the art. In some preferred embodiments, cells or tissues are homogenized in the presence of chaotropic agents to inhibit nuclease. In some other embodiments, RNase are inhibited or destroyed by heart treatment followed by proteinase treatment.

Methods of isolating total RNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993)).

In a preferred embodiment, the total RNA is isolated from a given sample using, for example, an acid guanidinium-phenol-chloroform extraction method and polyA+ mRNA is isolated by oligo dT column chromatography or by using (dT)n magnetic beads. (See, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York, 1987.) In one particularly preferred embodiment, total RNA is isolated from mammalian cells using RNeasy Total RNA isolation kit (QIAGEN). If mammalian tissue is used as the source of RNA, a commercial reagent such as TRIzol Reagent (GIBCOL Life Technologies). A second cleanup after the ethanol precipitation step in the TRIzol extraction using Rneasy total RNA isolation kit may be beneficial.

Hot phenol protocol described by Schmitt et al., (1990) Nucleic Acid Res., 18:3091-3092 is useful for isolating total RNA for yeast cells.

Good quality mRNA may be obtained by, for example, first isolating total RNA and then isolating the mRNA from the total RNA using Oligotex mRNA kit (QIAGEN).

Total RNA from prokaryotes, such as E. coli. Cells, may be obtained by following the protocol for MasterPure complete DNA/RNA purification kit from Epicentre Technologies (Madison, Wis.).

Frequently, it is desirable to amplify the nucleic acid sample prior to hybridization. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

Other suitable amplification methods include, but are not limited to polymerase chain reaction (PCR) (Innis et al., PCR Protocols. A guide to Methods and Application. Academic Press, Inc. San Diego, (1990)), ligase chain reaction (LCR). (SeeWu and Wallace, Genomics, 4: 560 (1989), Landegren et al., Science, 241: 1077 (1988) and Barringer et al., Gene, 89: 117 (1990), transcription amplification (Kwoh et al., Proc. Natl. Acad. Sci. USA, 86: 1173 (1989)), and self-sustained sequence replication (Guatelli et al., Proc. Nat. Acad. Sci. USA, 87: 1874, 1990.) Cell lysates or tissue homogenates often contain a number of inhibitors of polymerase activity. Therefore, RT-PCR typically incorporates preliminary steps to isolate total RNA or mRNA for subsequent use as an amplification template. One tube mRNA capture method may be used to prepare poly(A)+ RNA samples suitable for immediate RT-PCR in the same tube (Boehringer Mannheim). The captured mRNA can be directly subjected to RT-PCR by adding a reverse transcription mix and, subsequently, a PCR mix.

In a particularly preferred embodiment, the sample mRNA is reverse transcribed with a reverse transcriptase and a primer consisting of oligo dT and a sequence encoding the phage T7 promoter to provide a single stranded DNA template. The second DNA strand is polymerized using a DNA polymerase with or without primers. (See U.S. patent application Ser. No. 09/102,167, and U.S. Provisional Application Serial No. 60/172,340, both incorporated herein by reference for all purposes.) After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art. (See, e.g., Sambrook, supra.) and this particular method is described in detail by Van Gelder et al., Proc. Natl. Acad. Sci. USA, 87: 1663-1667, 1990. Moreover, Eberwine et al. Proc. Nati. Acad. Sci. USA, 89: 3010-3014 provide a protocol that uses two rounds of amplification via in vitro transcription to achieve greater than 106 fold amplification of the original starting material thereby permitting expression monitoring even where biological samples are limited. In one preferred embodiment, the in-vitro transcription reaction may be coupled with labeling of the resulting cRNA with biotin using Bioarray high yield RNA transcript labeling kit (Enzo P/N 900182).

Before hybridization, the resulting .cRNA may be fragmented. One preferred method for fragmentation employs RNase free RNA fragmentation buffer (200 mM tris-acetate, pH 8.1, 500 mM potassium acetate, 150 mM magnesium acetate). Approximately 20 μg of cRNA is mixed with 8 μL of the fragmentation buffer. RNase free water is added to make the volume to 40 μL. The mixture may be incubated at 94° C. for 35 minutes and chilled in ice.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid, pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lambda vectors designed for Cre-loxP plasmid subcloning. (See, e.g., Palazzolo et al., Gene, 88: 25-36, 1990.) The biological sample should contain nucleic acids that reflects the level of at least some of the transcripts present in the cell, tissue or organ of the species of interest. In some embodiments, the biological sample may be prepared from cell, tissue or organs of a particular status. For example, a total RNA preparation from the pituitary of a dog when the dog is pregnant. In another example, samples may be prepared from E. Coli cells after the cells are treated with IPTG. Because certain genes may only be expressed under certain conditions, biological samples derived under various conditions may be needed to observe all transcripts. In some instance, the transcriptional annotation may be specific for a particular physiological, pharmacological or toxicological condition. For example, certain regions of a gene may only be transcribed under specific physiological conditions. Transcript annotation obtained using biological samples from the specific physiological conditions may not be applicable to other physiological conditions.

Detection of Transcription Activities Using Microarrays

As used herein, the term “transcript” refers to RNA molecules which include molecules that are producted by RNA transcription and posttranscriptional modifications. Transcription activities may be stuided using nucleic acid hybridization. More particularly, a transcript may be detected by detecting the hybridization of a nucleic acid probe that can specifically hybridize with the transcript As used herein, a “probe” is a molecule for detecting or binding a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that these are illustrative embodiments that are not to limit the invention in any way.

In preferred embodiments, probes may be immobilized on substrates to create an array. An “array” may comprise a solid support with peptide or nucleic acid or other molecular probes attached to the support. Arrays typically comprise a plurality of different nucleic acids or peptide probes that are coupled to a surface of a substrate in different, known locations. These arrays, also described as “microarrays” or colloquially “chips” have been generally described in the art, for example, in Fodor et al., Science, 251:767-777 (1991), which is incorporated by reference for all purposes.

Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501 which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPS procedures. Using the VLSIPS M approach, one heterogeneous array of polymers is converted, through simultaneous coupling at a number of reaction sites, into a different heterogeneous array. See, U.S. Pat. Nos. 5,384,261 and 5,677,195.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743, 6,140,044 and D430024, all of which are incorporated by reference in their entireties for all purposes.

Methods for signal detection and processing of intensity data are additionally disclosed in, for example, U.S. Pat. Nos. 5,547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252,5,834,758, 5,837,832, 5,843,655, 5,856,086, 5,856,104, 5,856,174, 5,858,659,5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996 and 6,136,269, all of which are incorporated by reference in their entireties for all purposes.

In some embodiments, nucleic acid probes designed to detect transcripts from a region of a genome are hybridized with a nucleic acid sample derived from the species with the genome. Because either strand of the genomic DNA can serve as a template, probes that can detect the transcripts or nucleic acids dervied from the transcripts may be employed. Methods for deciphering which strand act as the template for a transcript are described in, for example, U.S. patent application Ser. No. 09/683,221, filed on Dec. 3, 2001, which is incorporated herein by reference for all purposes. In some embodiments, the actual sequences of the nucleic acid probes may be dependent upon the assay protocols. For example, if the transcripts are directly hybridized, the probes for detecting the transcripts should be complementary potential transcripts. Alternatively, if a sample derived from the transcripts, via, for example, reverses transcription or amplification, the probes should be complementary with the derived nucleic acids. The probes may be designed according to the reference sequence of a genome. In a particularly preferred embodiment, probe sequences are obtained from both strand of the genomic DNA so that potential transcripts from either strand can be detected.

While various aspects of the invention are primarily described using examplary embodiments which use high density oligonucleotide probes, this invention is not limited to any particular microarray format. For example, the probes may be presynthesized, and immobilized on beads or optical fibers.

The nucleic acid sample containing potential transcripts or nucleic acids derived from potential transcripts can be hybridized with the probes to detect whether a particular region of the genome is transcribed. One of skill in the art will appreciate that hybridization conditions may be selected to provide any degree of stringency. In a preferred embodiment, hybridization is performed at low stringency in this case in 6× SSPE-T at 37 C (0.005% Triton X-100) to ensure hybridization and then subsequent washes are performed at higher stringency (e.g., 1× SSPE-T at 37 C) to eliminate mismatched hybrid duplexes. Successive washes may be performed at increasingly higher stringency (e.g., down to as low as 0.25× SSPE-T at 37 C to 50 C) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

In a preferred embodiment, background signal is reduced by the use of a detergent (e.g., C-TAB) or a blocking reagent (e.g., sperm DNA, cot-1 DNA, etc.) during the hybridization to reduce non-specific binding. In a particularly preferred embodiment, the hybridization is performed in the presence of about 0.5 mg/ml DNA (e.g., herring sperm DNA). The use of blocking agents in hybridization is well known to those of skill in the art. (See, e.g., Chapter 8 in P. Tijssen, supra.) The stability of duplexes formed between RNAs or DNAs are generally in the order of RNA:RNA>RNA:DNA>DNA:DNA, in solution. Long probes have better duplex stability with a target, but poorer mismatch discrimination than shorter probes (mismatch discrimination refers to the measured hybridization signal ratio between a perfect match probe and a single base mismatch probe). Shorter probes (e.g., 8discriminate mismatches very well, but the overall duplex stability is low.

Altering the thermal stability (Tm) of the duplex formed between the target and the probe using, e.g., known oligonucleotide analogues allows for optimization of duplex stability and mismatch discrimination. One useful aspect of altering the Tm arises from the fact that adenine-thymine (A-T) duplexes have a lower Tm than guanine-cytosine (G-C) duplexes, due in part to the fact that the A-T duplexes have 2 hydrogen bonds per base-pair, while the G-C duplexes have 3 hydrogen bonds per base pair. In heterogeneous oligonucleotide arrays in which there is a non-uniform distribution of bases, it is not generally possible to optimize hybridization for each oligonucleotide probe simultaneously. Thus, in some embodiments, it is desirable to selectively destabilize G-C duplexes and/or to increase the stability of A-T duplexes. This can be accomplished, e.g., by substituting guanine residues in the probes of an array which form G-C duplexes with hypoxanthine, or by substituting adenine residues in probes which form A-T duplexes with 2,6 diaminopurine or by using the salt tetramethyl ammonium chloride (TMACI) in place of NaCl.

Altered duplex stability conferred by using oligonucleotide analogue probes can be ascertained by following, e.g., fluorescence signal intensity of oligonucleotide analogue arrays hybridized with a target oligonucleotide over time. The data allow optimization of specific hybridization conditions at, e.g., room temperature (for simplified diagnostic applications in the future).

Another way of verifying altered duplex stability is by following the signal intensity generated upon hybridization with time. Previous experiments using DNA targets and DNA chips have shown that signal intensity increases with time, and that the more stable duplexes generate higher signal intensities faster than less stable duplexes. The signals reach a plateau or “saturate” after a certain amount of time due to all of the binding sites becoming occupied. These data allow for optimization of hybridization, and determination of the best conditions at a specified temperature.

Methods of optimizing hybridization conditions are well known to those of skill in the art. (See, e.g., Laboratory Techniques in Biochemistry and Molecular Biology, Vol. 24: Hybridization With Nucleic Acid Probes, P. Tijssen, ed. Elsevier, N.Y., 1993.) In a preferred embodiment, the hybridized nucleic acids are detected by detecting one or more labels attached to the sample nucleic acids. The labels may be incorporated by any of a number of means well known to those of skill in the art. However, in a preferred embodiment, the label is simultaneously incorporated during the amplification step in the preparation of the sample nucleic acids. Thus, for example, polymerase chain reaction (PCR) with labeled primers or labeled nucleotides will provide a labeled amplification product. In a preferred embodiment, transcription amplification, as described above, using a labeled nucleotide (e.g., fluorescein-labeled UTP and/or CTP) incorporates a label into the transcribed nucleic acids. Alternatively, cDNAs synthesized using a RNA sample as a template, cRNAs are synthesized using the cDNAs as templates using in vitro transcription (IVT). A biotin label may be incorporated during the IVT reaction (Enzo Bioarray high yield labeling kit).

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. One particularly preferred method uses colloidal gold label that can be detected by measuring scattered light.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an aviden-conjugated fluorophore will bind the biotin bearing hybrid duplexes providing a label that is easily detected. For a detailed review of methods of labeling nucleic acids and detecting labeled hybridized nucleic acids. (See Laboratory Techniques in Biochemistry and Molecular Biology, Vol. 24: Hybridization With Nucleic Acid Probes, P. Tijssen, ed. Elsevier, N.Y., 1993.) Fluorescent labels are preferred and easily added during an in vitro transcription reaction. In a preferred embodiment, fluorescein labeled UTP and CTP are incorporated into the RNA produced in an in vitro transcription reaction as described above.

Means of detecting labeled target (sample) nucleic acids hybridized to the probes of the high density array are known to those of skill in the art. Thus, for example, where a colorimetric label is used, simple visualization of the label is sufficient. Where a radioactive labeled probe is used, detection of the radiation (e.g., with photographic film or a solid state detector) is sufficient.

In a preferred embodiment, however, the target nucleic acids are labeled with a fluorescent label and the localization of the label on the probe array is accomplished with fluorescent microscopy. The hybridized array is excited with a light source at the excitation wavelength of the particular fluorescent label and the resulting fluorescence at the emission wavelength is detected. In a particularly preferred embodiment, the excitation light source is a laser appropriate for the excitation of the fluorescent label.

In one aspect of the invention, methods are provided for detecting a transcribed genomic region. The methods include providing a nucleic acid sample containing transcripts or nucleic acids derived from transcripts from the genome; hybridizing the nucleic acid sample with a plurality of nucleic acid probes, where the probes are designed to interrogate potential transcripts from both strands of the genomic DNA; and analyzing hybridization signals to detect the transcribed region. Typically, a reference sequence for a genome is used for the selection of the probes. As used herein, the reference sequence of a genome is a genomic sequence that is available from public or private databases. Such a reference sequence may come from an individual genome or is a composite of several to many individual genomes. In some embodiments, probes tiling the reference sequence (and its complementary sequence) are selected. The probes can be at least 10, 15, 20, 25, 30, 35, 40, 45, 50, 55, or 60 bases in length. One of skill in the art would appreciate that the coverage of probes against a genomic region can vary. In some instances, the probes are selected at an interval of every 1, 10, 25, 50, 100, 500, 1000, or 200 bases. In some embodiments, the plurality of probes comprises probes interrogating the intergenic, and intronic regions of the genome. The probes may be immobilized on a substrate at a density greater than 400 or 1000 different probes per cm².

In another aspect of the invention, methods are provided for detecting an operon element in a prokaryote. The methods include hybridizing transcripts or nucleic acids dervied from transcripts from the organism with a plurality of probes, where the probes interrogate transcription of an intergenic region between two flanking open reading frames (ORFs); and classifying the intergenic region as a potential operon element if both flanking ORFs are expressed and if the intergenic region is transcribed off the same DNA strand as the flanking ORFs.

In some embodiments, the methods include classifying the intergenic region as operon element if both flanking ORFs are expressed and if the intergenic region Is transcribed off the same DNA strand as the flanking ORFs and if transcription in the intergenic region is detected by more than 60% or 80% of the probes targeting the intergenic region.

In some preferred embodiments, method include classifying the intergenic region as a potential operon element if both flanking ORFs are expressed and if the intergenic region is transcribed off the same DNA strand as the flanking ORFs and the transcription of the intergenic region is correlated with the transcription of at least one of the flanking ORFs.

In yet another aspect of the invention, methods for detecting untranslated region (UTR) for a gene are provided. The methods include hybridizing a sample containing transcripts or nucleic acids dervied from transcripts with a plurality of probes, where the probes interrogate transcription of an intergenic region immediately upstream the gene; and classifying the intergenic region as a potential 5″UTR of the gene if the intergenic region is transcribed in the same orientation of the gene and the trancribed region is greater than 70 bases in length. Similarly, an intergenic region is classified as a potential 3″UTR of the gene if the intergenic region is transcribed in the same orientation of the gene, it is immediately downstream of the gene and the trancribed region is greater than 70 bases in length.

### EXAMPLE

This example (See, Brian Tjaden, 2001, Transcriptome Analysis of Escherichia coli using High-Density Oligonucleotide Probe Arrays, Genes & Development, 15:1637, incorporated herein by reference for all purposes) shows the interrogation of the Escherichia coli MG1655 genome sequence for transcription activities and the identification of transcripts according to the exemplary embodiments of the invention. By interrogating both strands of a genome sequence on a microarray at a high resolution, RNA transcripts can be globally identified and linked back to the genome sequence, allowing more accurate annotation predictions. In this study, high-density oligonucleotide probe arrays on which the complete Escherichia coli MG1655 genome sequence is represented were used to identify RNA transcripts in the intergenic (Ig) regions. Each previously annotated open-reading frame (ORF) (Blattner, F. R. et al. The complete genome sequence of Escherichia coli K-12 [see comments]. Science 277, 1453-74 (1997)) has 15 oligonucleotide probes, which are designed to be complementary to the sense strand and each intergenic region greater than 40 bp is interrogated with 15 probes on each of the forward and reverse strands. Since microarrays traditionally interrogate only the in silico identified translated region of a gene, we consider all elements between translated regions as intergenic. The sequence of the oligonucleotide probes and their location in regards to the genome sequence have been published (arep.med.harvard.edu/ExpressDB/EDS37/GAPS_webpages/GAPS_main.htm, last visited on Feb. 2, 2002) and provide the basis for a detailed analysis of the E. coli transcriptome.

Materials and Methods E. coli strain MG1655 cells were grown in Luria-Bertani liquid or on solid medium and used for inoculation of liquid cultures. Cells were grown in 50-ml batch cultures in 250-ml Erlenmeyer flasks at 37° C. with aeration by rotary shaking (300The culture media used were Luria-Bertani (LB) or M9 minimal medium described elsewhere supplemented with glucose (0.2%) or glycerol (0.2%) (Sambrook, J., Fritsch, E. F. & Maniatis, T. Molecular Cloning (ed. Nolan, C.) (Cold Spring Harbor Press, Cold Spring Harbor, 1989)). Anaerobic growth was performed at 37° C. in the same flask fitted with butyl rubber stoppers and the air in the dead space replaced with argon. Growth was monitored at 600on a Hitachi U-2000 spectrophotometer. Cells were harvested in mid logarithmic phase (mid log), midway between logarithmic phase and stationary phase, early stationary phase or deep stationary growth phase (24 hours after the culture reached stationary phase)

The cDNA synthesis method was described previously (Rosenow, C., Saxena, R. M., Durst, M. & Gingeras, T. R. Prokaryotic RNA preparation methods, useful for high density array analysis: Comparison of two approaches. Nucleic Acid Research 29, e112 (2001)). Briefly, 10 μg of total RNA was reverse transcribed using the Superscript II system for first strand cDNA synthesis from Life Technologies (Rockville, Md.). The remaining RNA was removed using 2 U. RNase H (Life Technologies, Rockville, Md.) and 1 μg RNase A (Epicentre, Madison, Wis.) for 10 min at 37° C. in 100 μl total volume. The cDNA was purified using the Qiaquick PCR purification kit from Qiagen (Valencia, Calif.). Isolated cDNA was quantitated based on the absorption at 260 nm and fragmented using a partial DNase I digest. The fragmented cDNA was 3″ end-labeled using terminal transferase (Roche Molecular Biochemicals, Indianapolis, Ind.) and biotin-N6-ddATP (DuPont/NEN, Boston, Mass.). The fragmented and end-labeled cDNA was added to the hybridization solution without further purification.

5 μg of E. coli genomic DNA was fragmented using 0.2 U DNase I (Roche, Indianapolis, Ind.) in one-phor-all buffer (Amersham, Piscataway, N.J.), adjusted to a final volume of 20 μl and incubated at 37° C. for 10 minutes, followed by inactivation at 99° C. for 10 minutes. The fragmented DNA was subsequently labeled with terminal transferase (Roche, Indianapolis, Ind.) and biotin-N6-ddATP (DuPont/NEN, Boston, Mass.) in accordance with the manufacturer's protocol. Standard hybridization, wash, and stain protocols were used (Affymetrix, Santa Clara, Calif.).

The GeneChip(R) Software analysis program MAS 4.1 and DMT 2.0 (Affymetrix, Santa Clara, Calif.) were used for the analysis of gene expression and expression clustering, respectively. To identify transcripts within intergenic regions, we developed an algorithm for the analysis of the .ce/file, generated by MAS 4.1. The .ce/file contains the probe location and the individual intensities of the perfect match and the corresponding mismatch on the microarray. In order to identify transcripts, sets of adjacent probes (two or more probes) in which the PM-MM for each adjacent probe exceeds an expression threshold in both replicates (based on empirical results, a difference threshold of 200 was used) were examined. In this example, a strict criteria for transcript identification was used to ensure a high specificity for transcript detection. For each duplicate experiment, all possible transcripts which met these criteria in all interrogated Ig regions were searched. In order to correct for possible crosshybridization effects, labeling inconsistencies or hybridization variations, neighboring transcripts in the same Ig region were combined intq a single transcript if they were separated by a single probe which failed to meet our expression criteria. This approach was applied to all interrogated Ig regions genome-wide, and then proceeded to classify the identified transcripts into one of the following categories: operon elements, 5″ UTRs, 3″ UTRs and stand alone transcripts, which represent transcripts that did not fall into any of the previous categories.

Initial analysis of the data across all experiments showed a range of hybridization affinities for different probes. 2671 probes in the intergenic regions were removed from the analysis for which there was evidence of significant crosshybridization or other nonspecific hybridizations. These probes were determined by hybridizing E. coli genomic DNA labeled directly with terminal transferase to the probe array, and removing the probes, that failed to meet our difference threshold. The remaining probes were studied by hybridizing biotin labeled cDNA (Rosenow, C., Saxena, R. M., Durst, M. & Gingeras, T. R. Prokaryotic RNA preparation methods, useful for high density array analysis: Comparison of two approaches. Nucleic Acid Research 29, e112 (2001)) derived from 13 different growth conditions in duplicate for a total of 26 arrays.

A stringent difference model was developed for the transcript discovery. This was based on evidence that actual expression levels can be linearly approximated by such a model (Li, H. & Hong, F. Cluster-Rasch models for microarray gene expression data. Genome Biol 2 (2001); Lockhart, D. J. et al. Expression monitoring by hybridization to high-density oligonucleotide arrays. Nat Biotechnol 74, 1675-80. (1996)). A probe had to meet the difference requirement in both duplicate experiments before the probe is considered as “expressed”. After identifying a conservative set of potential transcripts in Ig regions, they were classified based on their genome location as operon elements, 5-prime untranslated regions (5″ UTRs), 3-prime UTRs or as transcripts with unknown function. For additional validation of the classification, the co-regulation of the identified transcripts with their flanking ORFs using the self-organizing map (SOM) algorithm was determined (Tamayo, P. et al. Interpreting patterns of gene expression with self-organizing maps: methods and application to hematopoietic differentiation. Proc Natl Acad Sci USA 96, 2907-12. (1999)). Transcripts that are co-regulated across many conditions are likely to be from the same transcript. In addition, homology search against the complete genome sequence of Salmonella typhi (the closest fully-sequenced relative to E. coli) was conducted to identify conserved regions (Altschul, S. F. et al. Gapped BLAST and PSI-BLAST: a new generation of protein database search programs. Nucleic Acids Res 25, 3389-402. (1997)). Sequences can be conserved for many different reasons, including coding regions, complex promoters or leader sequences, transcriptional and post-transcriptional regulatory signals, small RNAs, transcriptional terminators, and sequences of as yet unknown function. The cluster and homology analysis were used together with annotation programs (Salzberg, S. L., Delcher, A. L., Kasif, S. & White, O. Microbial gene identification using interpolated Markov models. Nucleic Acids Res 26, 544-8. (1998)) and information collected from the literature and public databases to further characterize the transcripts and to classify them as potential new ORFs or RNA transcripts that serve as small regulatory RNA (sRNA).

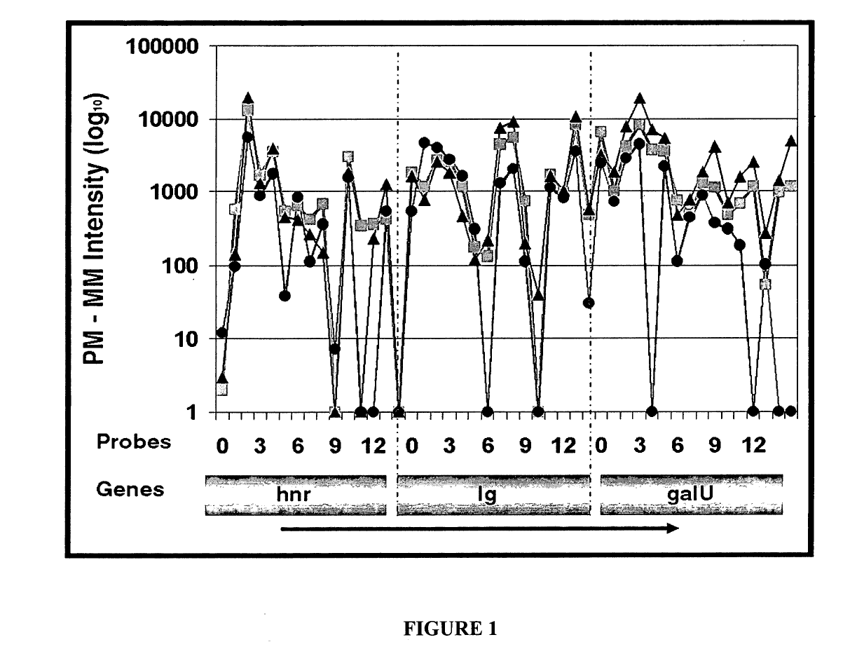

Ig transcripts are classified as operon elements if both flanking ORFs are expressed, if the Ig region is transcribed off the same DNA strand as the flanking ORFs and if the expressed transcript extends across the entire Ig region, except possibly isolated single probes. To improve sensitivity, we allow up to one probe in a probe set not to be expressed. Using these criteria, 293 transcripts and their flanking genes were identified as operon elements. 289 of these Ig regions have been previously documented or predicted as being part of an operon (http://www.cifn.unam.mx/Computational_Genomics/GETools/E. coli-predictions.html). Based on this comparison the false positive rate for transcript detection was estimated to be less than 1%. Cluster analysis revealed that 71% of the previously predicted operons showed co-regulation of at least two out of three transcripts (flanking genes and Ig region) while 81% of the documented operons offered this evidence of co-regulation. When co-regulation for all three transcripts was required, 17% of the predicted operons showed evidence compared to 44% of the documented operons. FIG. 1 shows the expression levels for individual probes interrogating the predicted hnr-galU operon. RT-PCR confirmed a single RNA transcript for these two genes and the Ig region (data not shown). Six additional operons were experimentally confirmed using RT-PCR (Table 3, supplemental data). From a total of 931 predicted and documented operons in Regulon DB (21) which meet our criteria for being operon elements, we detect 334 using our microarray analysis. This results in a false negative rate of less than 64%. This unusual high false negative rate is consistent with the fact that we use a very conservative transcript prediction model and in addition the majority of the operons listed in Regulon DB are predicted operons without experimental validation. Two Ig regions that have not been reported to be part of an operon were found to be co-regulated either with both flanking genes (C0794: rpsM/rpmJ) or with the downstream gene (C0789: rplN/rpsQ). Both Ig regions are flanked on one side by documented operons containing genes for 30S and 50S ribosomal subunit proteins and on the other side with a gene encoding a 50S ribosomal subunit protein. Based on our findings and the close functional relationship of the gene products, they are strong candidates for new, previously unidentified operons. The third potential operon candidate (C0669; nLpD/pcm) was found to have co-regulated flanking genes. The two genes have no obvious functional relationships and need to be further analyzed. The fourth operon candidate (C0064: yaeD/rrsH) shows no co-regulation with the flanking genes and is located between a gene with unknown function and the 16S RNA of the rrnH operon.

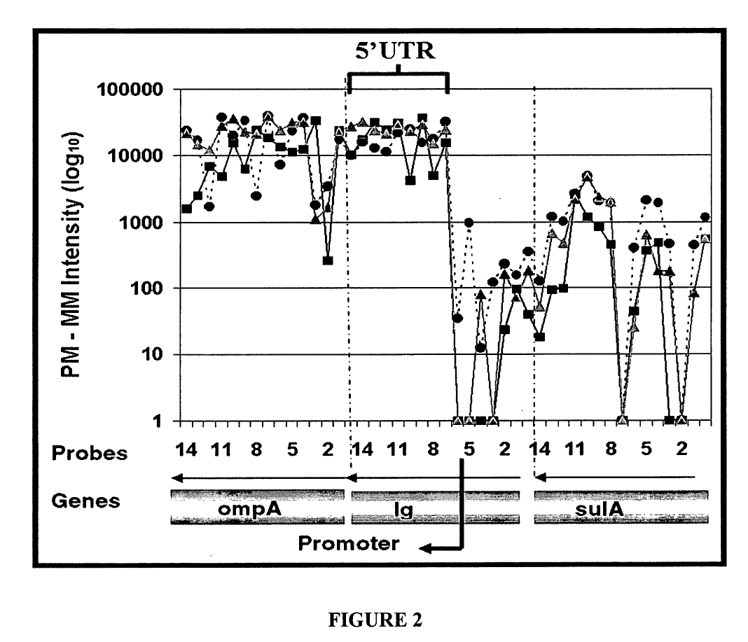

As with the operons described above, experimental evidence for 5-prime expressed regions can supplement computational approaches by identifying not only transcription start sites for genes, but also multiple start sites when different promoters are employed under different conditions as well as cis-regulatory sites upstream of known genes. In order for an Ig transcript to be classified as a 5″ UTR in our analysis, we required the transcript to be in the same orientation as its downstream gene and to be expressed under the same growth conditions. We assume that the transcript should be ≧70 nucleotides (nt) to encode a 5″ UTR, slightly longer than the expected 50 60 nts of a promoter and that the transcript extends close to the downstream genes translational start site, i.e., the transcript should extend to the penultimate or ultimate probe in the probe set of the. Ig region. FIG. 2 shows an example for the transcribed but not translated leader sequence of the ompA mRNA (Chen, L. H., Emory, S. A., Bricker, A. L., Bouvet, P. & Belasco, J. C. Structure and function of a bacterial mRNA stabilizer: analysis of the 5′ untranslated region of ompA mRNA. J Bacteriol 773, 4578-86. (1991)). The PM minus MM probe intensities and the probe locations were used to determine the transcriptional start site, which was found to be close to the predicted promoter location for the ompA gene. A conservative set of 353 transcripts which met our expression criteria for 5″ UTRs were identified. 294 of these transcripts either showed concordant expression with their downstream ORF in all 13 experiments or else showed homology to Salmonella typhiwith an E-value <0.01 (Altschul, S. F. et al. Gapped BLAST and PSI-BLAST: a new generation of protein database search programs. Nucleic Acids Res 25, 3389-402. (1997)) and an overall identity of >65%. Fifteen 5″ UTRs contain conserved regulatory sequences, (http://www.cifn.unam.mx/Computational_Genomics/GETools/E.coll-predictions.html), two match previously identified small RNAs (sraB, crpT) (Rivas, E., Klein, R. J., Jones, T. A. & Eddy, S. R. Computational identification of noncoding RNAs in E. coli by comparative genomics. Curr Biol 11, 1369-73. (2001); Wassarman, K. M., Repoila, F., Rosenow, C., Storz, G. & Gottesman, S. Identification of novel small RNAs using comparative genomics and microarrays. Genes Dev 15, 1637-51. (2001); Argaman, L. et al. Novel small RNA-encoding genes in the intergenic regions of Escherichia coli. Curr Biol 11, 941-50. (2001)) and 49 transcripts fall into potential small ORF regions.

The classification of transcripts as 3-prime UTRs is analogous to that of the 5′ UTRs. The Ig transcript is in the same orientation as its upstream gene and expressed under the same growth conditions. In addition, we restricted the transcripts to be at least 70 bp in length, and to extend close to the upstream gene's predicted translational stop site. According to this criteria, 122 potential 3″ UTRs were identified, of which 69% are either concordantly expressed with their upstream gene in all 13 experiments or have sequence homology to Salmonella typhiwith an E-value of <0.01 and an overall identity of >65% (Table 5, supplemental data). Eleven of the 122 transcripts fell into potential small ORF regions.

Finally, 334 transcripts longer than 70 bp were identified. The transcripts were expressed according to the criteria but that could not be classified as operon elements, 5″ UTRs or 3″ UTRs based on the specific criteria for this example. This group of transcripts has a hybridization signal separate from and discontinuous with the signals from neighboring ORFs. Over 200 transcripts in this group showed sequence homofogywith Salmonella typhior considerable expression levels (more than 3 times background). This group also contains 17 known sRNA transcripts and 31 potential new ORF regions.

The above description is illustrative and not restrictive. Many variations of the invention will become apparent to those of skill in the art upon review of this disclosure. Merely by way of example a variety of substrates, receptors, ligands, and other materials may be used without departing from the scope of the invention. The scope of the invention should, therefore, be determined not with reference to the above description, but instead should be determined with reference to the appended claims along with their full scope of equivalents.

1. A method for identifying a transcribed genomic region comprising:
providing a nucleic acid sample comprising transcripts or nucleic acids dervied from transcripts from the genome;
hybridizing the nucleic acid sample with a plurality of nucleic acid probes, wherein the probes are designed to interogate potential transcripts from both strands of the genomic DNA;
analyzing hybridization signals to detect the transcribed region.

2. The method of claim 1 where the pluarlity of probes comprises probes interogating the intergenic, and intronic regions of the genome.

3. The method of claim 2 wherein the plurality of nucleic acid probes are immobilized on a substrate at a density greater than 400 different probes per cm².

4. The method of claim 3 wherein the plurality of nucleic acid probes are immobilized on on a substrate at a density greater than 1000 different probes per cm².

5. A method for detecting an operon element in a prokaryote organism comprising;
hybridizing transcripts or nucleic acids dervied from transcripts from the organism with a plurality of probes, wherein the probes interrogate transcription of an intergenic region between two flanking open reading frames (ORFs); and
classifying the intergenic region as a potential operon element if both flanking ORFs are expressed and if the intergenic region is transcribed off the same DNA strand as the flanking ORFs.

6. The method of claim 5 wherein the probes are oligonucleotides immobilized on a substrate.

7. The method of claim 6 wherein the probes are immobilized at a density of at least 400 different probes per cm².

8. The method of claim 7 wherein the plurality of nucleic acid probes are immobilized on on a substrate at a density greater than 1000 different probes per cm².

9. The method of claim 8 wherein ciassifying the intergenic region as operon element if both flanking ORFs are expressed and if the intergenic region is transcribed off the same DNA strand as the flanking ORFs and if transcription in the intergenic region is detected by more than 60% of the probes targeting the intergenic region.

10. The method of claim 9 wherein the intergenic region is detected by more than 80% of the probes targeting the intergenic region.

11. The method of claim 9 wherein the classifying comprises classifying the intergenic region as a potential operon element if both flanking ORFs are expressed and if the intergenic region is transcribed off the same DNA strand as the flanking ORFs and the transcription of the intergenic region is correlated with the transcription of at least one of the flanking ORFs.

12. The method of claim 11 wherein the transcription of the intergenic region is correlated with the transcription of both flanking ORFs.

13. A method for detecting a 5″ UTR for a gene comprising;
hybridizing a sample comprising transcripts or nucleic acids dervied from transcripts with a plurality of probes, wherein the probes interrogate transcription of an intergenic region immediately upstream the gene; and
classifying the intergenic region as a potential 5″UTR of the gene if the intergenic region is transcribed in the same orientation of the gene and the trancribed region is greater than 70 bases in length.

14. The method of claim 13 wherein the probes are oligonucleotides immobilized on a substrate.

15. The method of claim 14 wherein the probes are immobilized at a density of at least 400 different probes per cm².

16. The method of claim 15 wherein the plurality of nucleic acid probes are immobilized on on a substrate at a density greater than 1000 different probes per cm².

17. A method for detecting a 3″ UTR for a gene comprising;
hybridizing a sample comprising transcripts or nucleic acids dervied from transcripts with a plurality of probes, wherein the probes interrogate transcription of an intergenic region immediately down stream the gene; and
classifying the intergenic region as a potential 3″UTR of the gene if the intergenic region is transcribed in the same orientation of the gene and the trancribed region is greater than 70 bases in length.

18. The method of claim 17 wherein the probes are oligonucleotides immobilized on a substrate.

19. The method of claim 18 wherein the probes are immobilized at a density of at least 400 different probes per cm².

20. The method of claim 19 wherein the plurality of nucleic acid probes are immobilized on on a substrate at a density greater than 1000 different probes per cm².
